(19) United States
(12) Patent Application Publication
(10) Pub. No.: US 2011/0306840 A1
(43) Pub. Date: Dec. 15, 2011

(54) CONTROLLABLE MAGNETIC SOURCE TO FIXTURE INTRACORPOREAL APPARATUS.

(75) Inventors: Peter K. Allen, Pleasantville, NY (US); Roger Goldman, New York, NY (US)

(73) Assignee: THE TRUSTEES OF COLUMBIA UNIVERSITY IN THE CITY OF NEW YORK, New York, NY (US)

(21) Appl. No.: 13/147,097

(22) PCT Filed: Jan. 29, 2010
(86) PCT No.: PCT/US10/22532

§ 371 Date: Sep. 1, 2011

Related U.S. Application Data
(60) Provisional application No. 61/148,730, filed on Jan. 30, 2009.

(51) Int. Cl. A61B 1/32

(52) U.S. Cl. 600/202

(57) ABSTRACT

A first magnetic field can be produced across a tissue region using a first magnetic field source, providing a magnetic coupling force between the first magnetic field source and a first object, wherein the first object provides a magnetic field or a magnetic susceptibility to obtain the magnetic coupling force. The magnetic coupling force can be sensed using a force sensor and a resulting sensed force signal can be provided to a controller. The controller can provide an output signal to control the magnetic coupling force using the sensed forced signal to obtain a constant or desired magnetic coupling force.

### TECHNICAL FIELD

This document pertains generally to medical devices, and more particularly, but not by way of limitation, to a controllable magnetic source for fixturing an intracorporeal apparatus.

### BACKGROUND

Recent advancements in surgical techniques provide for less-invasive (sometimes referred to as “minimally invasive) medical procedures, such as surgical procedures having smaller incisions into the body of a subject. Endoscopy generally includes a minimally invasive medical procedure that can be used to access an interior surface of an organ, such as by inserting a tube into the body of the subject via a small surgical incision or a bodily orifice. One form of endoscopy includes laparoscopy. Laparoscopy typically includes an operation in the abdomen that can be performed using a small incision (e.g., 0.5 cm, 1 cm, 1.5 cm, etc.) into the body. The incision location can be referred to as a trocar point. A trocar can include a hollow or three-sided surgical apparatus, through which a laparoscopic apparatus can be passed into the body. One type of laparoscopic apparatus can include a camera. In an example, the camera can be inserted into the abdominal cavity to allow a clinician to view the internal organs of the subject. In other examples, the laparoscopic apparatus can include other surgical instruments, such as a scalpel, a scissors, etc.

### OVERVIEW

The present inventors have recognized, among other things, that it is desirable to anchor the laparoscopic (or intracorporeal) apparatus at a desired location within the body to assist in a medical procedure.

A first magnetic field can be produced across a tissue region using a first magnetic field source, providing a magnetic coupling force between the first magnetic field source and a first object, wherein the first object provides a magnetic field or a magnetic susceptibility to obtain the magnetic coupling force. The magnetic coupling force can be sensed using a force sensor and a resulting sensed force signal can be provided to a controller. The controller can provide an output signal to control the magnetic coupling force using the sensed forced signal to obtain a constant or desired magnetic coupling force.

In Example 1, a system includes a first magnetic field source configured to produce a first magnetic field across a tissue region, the first magnetic field providing a magnetic coupling force between the first magnetic field source and a first object, a force sensor configured to sense the magnetic coupling force and to provide a resulting sensed force signal, and a controller configured to receive the sensed force signal and to provide in response an output signal for controlling the magnetic coupling force to obtain a desired magnetic coupling force.

In Example 2, the system of Example 1 optionally includes the first object, the first object including a magnetic field source or receiver configured to provide a magnetic field or a magnetic susceptibility to obtain the magnetic coupling force.

In Example 3, the first object of any one or more of Examples 1-2 optionally includes or is coupled to an intracorporeal apparatus.

In Example 4, the first magnetic field source of any one or more of Examples 1-3 optionally includes a first electromagnet configured to produce the first magnetic field.

In Example 5, the output signal of any one or more of Examples 1-4 optionally is configured to adjust the first magnetic field produced by the first electromagnet to obtain the desired magnetic coupling force.

In Example 6, the first magnetic field source of any one or more of Examples 1-5 optionally includes a first permanent magnet.

In Example 7, the output signal of any one or more of Examples 1-6 optionally is configured to control a distance between the first magnetic field source and the first object to obtain the desired magnetic coupling force.

In Example 8, the system of any one or more of Examples 1-7 optionally includes a mount configured to suspend the first magnetic field source near the tissue region.

In Example 9, the mount of any one or more of Examples 1-8 optionally is configured to use at least part of the force sensor to suspend the first magnetic field source near the tissue region.

In Example 10, the force sensor of any one or more of Examples 1-9 optionally includes a strain gauge.

In Example 11, the mount of any one or more of Examples 1-10 optionally is configured to obtain the desired magnetic coupling force by using the output signal to adjust a distance between the first magnetic field source and the first object.

In Example 12, the first magnetic field source of any one or more of Examples 1-11 optionally is configured to hold the first object to a location on tissue region using the desired magnetic coupling force.

In Example 13, the controller of any one or more of Examples 1-12 optionally is configured to adjust the output signal to obtain the desired magnetic coupling force across a plurality of different tissue thicknesses.

In Example 14, a method includes producing a first magnetic field across a tissue region using a first magnetic field source, providing a magnetic coupling force between the first magnetic field source and a first object using the first magnetic field, the first object providing a magnetic field or providing a magnetic susceptibility to obtain the magnetic coupling force, sensing the magnetic coupling force and providing a resulting sensed force signal, and controlling the magnetic coupling force using the sensed forced signal to obtain a desired magnetic coupling force.

In Example 15, the providing the magnetic coupling force between the first magnetic field source and the first object of Example 14 optionally includes providing a magnetic coupling force between the first magnetic field source and an intracorporeal apparatus.

In Example 16, the producing the first magnetic field using the first magnetic field source of any one or more of Examples 14-15 optionally includes using a first electromagnet.

In Example 17, the controlling the magnetic coupling force of any one or more of Examples 14-16 optionally includes adjusting the first magnetic field produced by the electromagnet to obtain the desired magnetic coupling force.

In Example 18, the producing the first magnetic field using the first magnetic field source of any one or more of Examples 14-17 optionally includes using a first permanent magnet.

In Example 19, the controlling the magnetic coupling force of any one or more of Examples 14-18 optionally includes adjusting a distance between the first magnetic field source and the first object to obtain the desired magnetic coupling force.

In Example 20, the sensing the magnetic field source of any one or more of Examples 14-19 optionally includes suspending the first magnetic field source near the tissue region using a strain gauge.

In Example 21, the method of any one or more of Examples 14-20 optionally includes fixing the first object to a location on the tissue region using the magnetic coupling force.

In Example 22, the controlling the magnetic coupling force to obtain the desired magnetic coupling force of any one or more of Examples 14-21 optionally includes maintaining the desired magnetic coupling force across a plurality of different tissue thicknesses.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1-3 illustrate generally examples of a system including a first magnetic field source, a force sensor, and a controller.

FIG. 4 illustrates generally an example of a method including controlling a magnetic coupling force between a first magnetic field and a first object using a sensed force signal to obtain a desired magnetic coupling force.

FIGS. 5A-5B illustrate generally examples of force relationships between three types of electromagnets and two types of fixed rare-earth magnets.

FIG. 6 illustrates generally an example of a relationship between an attraction force of magnets across varying separation distances through air and through tissue.

### DETAILED DESCRIPTION

Generally, an intracorporeal apparatus (including an intracorporeal portion of a laparoscopic apparatus) is located within a body of a human or animal subject, where it can be anchored or supported to assist in a medical procedure. In an example, the intracorporeal apparatus (e.g., a laparoscopic apparatus or other object) can be magnetically coupled to an external apparatus. The magnetic coupling can be used to hold or otherwise fix the intracorporeal apparatus to a desired or fixed position, such as by controlling a magnetic field source at one of the external or intracorporeal locations. The magnetic coupling force between the external apparatus and the intracorporeal apparatus can be controlled (e.g., using a force sensor) so as to obtain a constant or other desired magnetic coupling force to provide a physical force between the external apparatus and the intracorporeal apparatus. In an example, the desired magnetic coupling force can be obtained over a wide range of variations of the thickness of a medium (e.g., a tissue region) between the external apparatus and the intracorporeal apparatus. In certain examples, the desired magnetic coupling force can be specified such that it is strong enough to secure the intracorporeal apparatus to a fixed or desired position (e.g., a fixed position on a tissue region such as the abdominal wall), weak enough to not harm the tissue region between the intracorporeal apparatus and the external apparatus (e.g., by ceasing blood supply or otherwise supplying too much force to the tissue region), or both

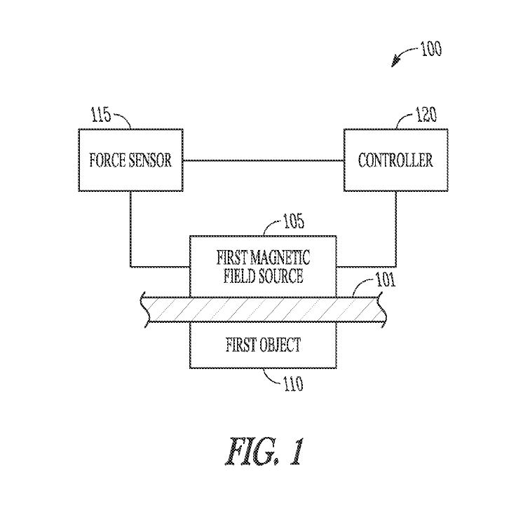

FIG. 1 illustrates generally an example of a system 100 including a first magnetic field source 105, a force sensor 115, and a controller 120. In an example, the system 100 can include a first object 110, separated from the first magnetic field source 105 by a tissue region 101.

In an example, the first magnetic field source 105 can include any material capable of producing a magnetic field. In certain examples, the first magnetic field source 105 can include at least one of an electromagnet, a permanent magnet, or other material capable of producing a magnetic field. In various examples, the first magnetic field source 105 can include a magnetic field source configured to be located proximate a tissue region (e.g., tissue region 101) either within or outside a body.

In an example, the first object 110 can include a magnetic field source or receiver configured to provide or receive a magnetic field or to provide a magnetic susceptibility to obtain a magnetic coupling force, such as an electromagnet or a magnetic material (e.g., a permanent magnet, a ferromagnetic material, or other magnetic material). In various examples, the first object 110 can include an object configured to be located proximate to a tissue region (e.g., tissue region 101) either within or outside a body.

In an example, the first object 110 can include or be coupled to an intracorporeal apparatus, such as an intracorporeal camera, scalpel, scissors, pliers, vacuum, or other surgical or medical apparatus. In other examples, the first magnetic field source 105 can include or be coupled to an intracorporeal apparatus. Generally, the first magnetic field source 105 and the first object 110 can be configured to provide a fixed or stationary support point for the intracorporeal apparatus.

In the example of FIG. 1, the first magnetic field source 105 can be configured to be located external to the body near the tissue region 101 and the first object 110 can be configured to be located internal to the body near the tissue region 101.

In an example, the first magnetic field can be coupled to the force sensor 115. The force sensor 115 can include any sensor configured to sense a magnetic coupling force between two objects (e.g., the first magnetic field source 105 and the first object 110) and to provide a resulting sensed force signal. In an example, the magnetic coupling force between the first magnetic field source 105 and the first object 110 can be adjusted to control the amount of force applied to the tissue region (e.g., so as to not harm the tissue region 101). In certain examples, the adjusting can include using information from the force sensor 115 (e.g., the sensed force signal) to measure the actual force, so that the adjusting can provide the desired actual force. In an example, the force sensor 115 can include a material (e.g., a semiconductor or other material) having at least one characteristic, property, or parameter (e.g., a resistance or other characteristic, property, or parameter) that changes depending upon the position, orientation, deformation, or other change of the material. In certain examples, the resulting sensed force signal can include the at least one characteristic, property, parameter, or other information from the force sensor 115.

In an example, the force sensor 115 can include a strain gauge. The strain gauge can include any device configured to measure deformation or strain. In an example, the strain gauge can include a flexible conductive foil pattern placed on a surface of a substrate material. In certain examples, the substrate material can include a metal, a plastic, or other material capable of supporting a load and withstanding a desired amount of deformation without being permanently affected by such deformation. In an example, the desired amount of deformation can include an amount that maintains the structural integrity of the substrate material (e.g., still supporting the load) but also deforms to an extent measurable by the strain gauge. In an example, as the substrate material flexes, bends, or otherwise deforms, the electrical properties of the strain gauge (e.g., the resistance, the capacitance, etc.) can change and this change can be measured. Thus, because the flex, bend, or deformation of the material can be indicative of the amount of force applied to the material, the force can be measured using the change of the electrical property of the strain gauge.

In an example, the force sensor 115 can include a pressure sensor (e.g., a piezoresistive material or other pressure sensor) capable of sensing a pressure that can be translated into a force between two objects, such as the first magnetic field source 105 and a first object 110. In an example, the pressure sensor can placed between at least one of the first magnetic field source 105 and the tissue region 101, or between the first object 110 and the tissue region 101. The amount of physical pressure sensed between the first magnetic field source 105 and the tissue region 101, between the first object 110 and the tissue region 101, or between the first magnetic field source 105 and the first object 110 can be indicative of the magnetic coupling force between the first magnetic field source 105 and the first object 110.

In certain examples, the force sensor 115 can include one or more other sensors, such as an optical or other sensor configured to sense the amount of strain, deformation, or other movement of the tissue region 101. In other examples, the force sensor can include one or more other sensors configured to sense the amount of strain, deformation, or other movement of a material coupled to at least one of the first magnetic field source 105 or the first object 110.

In an example, the force sensor 115 can include a blood flow sensor configured to sense the flow of blood through the tissue region 101 (e.g., the tissue region between the first magnetic field source 105 and the first object 110). In an example, the blood flow sensor can sense a reduction or stoppage in blood flow through the tissue region due to the pressure applied as a result of the magnetic coupling force between the first magnetic field source 105 and the first object 110. In this way, the measured blood flow can be used as a proxy to provide an indirect indication of the applied force. In various examples, a certain reduction or stoppage in blood flow through the tissue region 101 can be tolerated (e.g., indefinitely or for a certain period of time). Therefore, in certain examples, the measured blood flow can be monitored repeatedly during the duration of the procedure, such as to ensure that a specified tolerable limit has not been exceeded, thereby avoiding tissue necrosis or other potentially harmful consequences of the applied force.

In an example, at least one of the force sensor 115 or the first magnetic field source 105 can be coupled to the controller 120. The controller 120 can include a processor (e.g., central processing unit (CPU), microprocessor, or other processor), analog or digital circuit, or other controller (e.g., microcontroller, etc.). The controller 120 can be configured to receive information from the force sensor 115 (e.g., the sensed force signal) and to provide, in response to the received information, an output signal for controlling the magnetic coupling force between the first magnetic field source 105 and the first object 110. In an example, the magnetic coupling force can be controlled, such as to obtain or maintain a desired value of the magnetic coupling force.

In certain examples, the desired value of the magnetic coupling force can be specified such that it is strong enough to secure the intracorporeal apparatus to a fixed or desired position (e.g., a fixed position on a tissue region such as the abdominal wall), weak enough to not harm the tissue region between the intracorporeal apparatus and the external apparatus (e.g., by ceasing blood supply or otherwise supplying too much force to the tissue region), or both

In other examples, the desired magnetic coupling force can include a programmable or otherwise specifiable task-dependent coupling force. In an example, the magnetic coupling force required to maintain a fixed position for one activity or using a first instrument or apparatus can be more or less than the required force to maintain a fixed position for another activity or using a second instrument or apparatus. In other examples, the desired magnetic coupling force can be specified at a first value to secure the first object 110, and specified a second value to move (or permit movement of) the first object 110.

In an example, the controller 120, the force sensor 115, the first magnetic field source 105, and the first object 110 can operate as a feedback system (e.g., a closed-loop feedback system) to control the magnetic coupling force between the first magnetic field source 105 and the first object 110. In various examples, the tissue region 101 can include regions of varying thickness (e.g., different locations on a subject, or the same or different general location on different subject). For example, the tissue thickness of an abdominal wall of a child can be different than the tissue thickness of an abdominal wall of an adult. As another example, the tissue thickness of an abdominal wall of an obese adult can be different than that of an average adult. Accordingly, the first magnetic field can be adjusted, using the measured indication of force, to obtain the desired magnetic coupling force in tissue regions having an unknown or varying thickness.

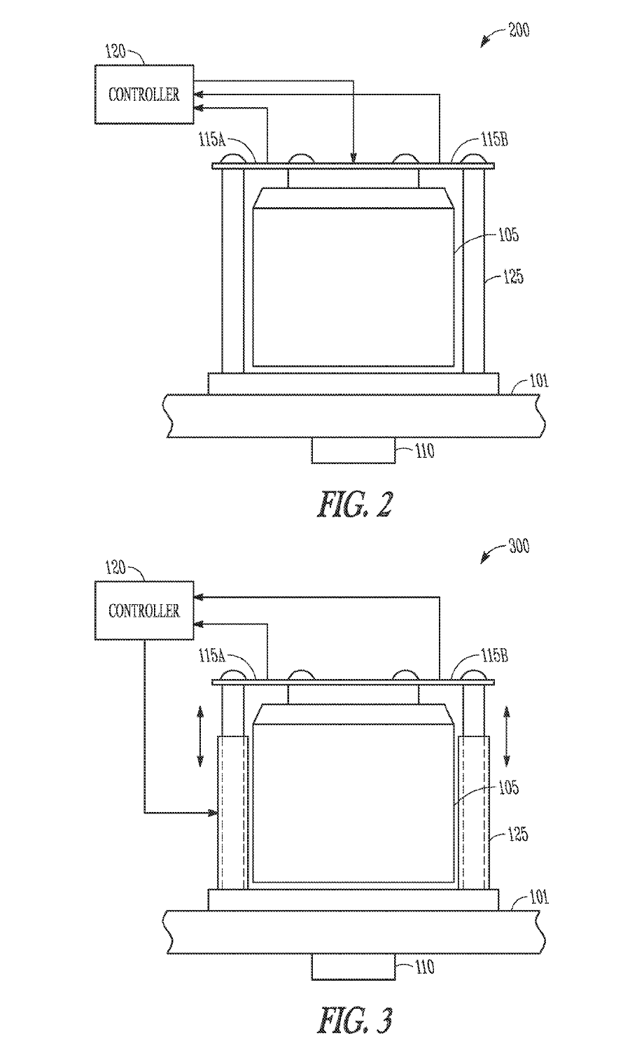

FIG. 2 illustrates generally an example of a system 200 including a first magnetic field source 105, a force sensor 115 (e.g., 115a or 115b), and a controller 120. In an example, the system 200 can include a first object 110, separated from the first magnetic field source 105 by a tissue region 101, and a housing 125.

In the example of FIG. 2, the first magnetic field source 105 can include an electromagnet and the first object 110 can include a magnetic material (e.g., a permanent magnet, a ferromagnetic material, or other magnetic material). In an example, the electromagnet can be coupled to the force sensor 115 (e.g., force sensor 115a or 115b), and the force sensor 115 can be coupled to the housing 125. In this example, the housing 125 can be configured to use the force sensor 115 to suspend the electromagnet near the tissue region 101. In an example, the force sensor 115 can include a strain gauge. As the magnetic coupling force between the electromagnet and the first object 110 increases, the amount of flex, bend, or deformation of the strain gauge can increase. Thus, the magnetic coupling force between the electromagnet and the first object 110 can be sensed using the strain gauge.

In an example, the first magnetic field source 105 and the force sensor 115 (e.g., force sensor 115a or 115b) can be coupled to the controller 120. The controller 120 can be configured to receive information from the force sensor 115 and provide an output signal for controlling the magnetic coupling force between the first magnetic field source 105 (e.g., the electromagnet) and the first object 110. In an example, the output signal from the controller 120 can be configured to adjust the first magnetic field produced by the first magnetic field source 105 (e.g., the electromagnet), such as by adjusting a current or other signal characteristic (e.g., pulse width, frequency, etc.) provided to the first magnetic field source 105. In an example, the first magnetic field can be adjusted to obtain a desired magnetic coupling force. The controller 120 can be further configured to provide a desired time-domain or frequency domain response for adjustably controlling the magnetic force by controlling the applied magnetic field in response to the sensed force. For example, controller can be configured to provide an over-damped response, an under-damped response, or a critically-damped response, as desired.

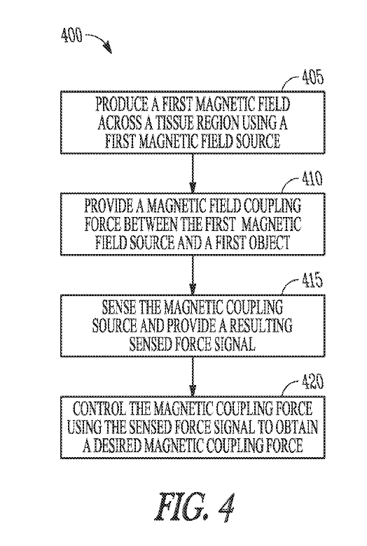

FIG. 3 illustrates generally an example of a system 300 including a first magnetic field source 105, a force sensor 115 (e.g., 115a or 115b), and a controller 120. In an example, the system 300 can include a first object 110, separated from the first magnetic field source 105 by a tissue region 101, and a housing 125.

In the example of FIG. 3, the first magnetic field source 105 can include a first permanent magnet and the first object 110 can include a magnetic material (e.g., a permanent magnet, a ferromagnetic material, or other magnetic material). In an example, the first permanent magnet can be coupled to the force sensor (e.g., force sensor 115a or 115b), and the force sensor 115 can be coupled to the housing 125. In this example, the housing 125 can be configured to suspend the first permanent magnet above the tissue region 101 using, the force sensor 115. In an example, the force sensor 115 can include a strain gauge. As the magnetic coupling force between the electromagnet and the first object 110 increases, the amount of flex, bend, or deformation of the strain gauge increases. Thus, the magnetic coupling force between the electromagnet and the first object 110 can be sensed using the strain gauge.

In an example, the housing 125 can be configured to adjust the distance between the first magnetic field source 105 and the first object 110 (e.g., by raising or lowering the permanent magnet). For example, the magnetic coupling force between the first magnetic field source 105 and the first object 110 can be adjusted by raising or lowering the first magnetic field source 105.

In an example, the first magnetic field source 105 and the force sensor 115 (e.g., force sensor 115a or 115b) can be coupled to the controller 120. The controller 120 can be configured to receive information from the force sensor 115 and to provide an output signal for controlling the magnetic coupling force between the first magnetic field source 105 (e.g., the first permanent magnet) and the first object 110. In an example, the output signal from the controller 120 can be configured to adjust the distance between the first magnetic field source 105 (e.g., the permanent magnet) and the first object 110, such as by using a raising or lowering mechanism of the housing 125. In an example, the distance between the first magnetic field source 105 and the first object 110 can be controlled or adjusted to obtain a desired magnetic coupling force.

In other examples, the housing 125 can be configured to adjust the distance between the first magnetic field source 105 and the first object 110 (e.g., by raising or lowering an electromagnet or the first object 110).

FIG. 4 illustrates generally an example of a method 400 including controlling a magnetic coupling force between a first magnetic field and a first object using a sensed force signal to obtain a desired magnetic coupling force. Generally, the thickness of a tissue region can vary from one location to another. Moreover, the thickness of a tissue region on one subject can be different than a tissue region on another subject (e.g., the thickness of a tissue region of a child can be different than that of an adult, the thickness of a tissue region of a healthy adult can be different than that of an unhealthy or obese adult, etc.). To accommodate for such variations in tissue thickness, the magnetic coupling force between the first magnetic field source and the first object can be controlled.

At 405, a first magnetic field across a tissue region (such as tissue region 101) is produced using a first magnetic field source. In an example, the first magnetic field source can include the first magnetic field source 105 (e.g., a first electromagnet or a first permanent magnet).

At 410, a magnetic coupling force between the first magnetic field source and a first object can be provided. In certain examples, the first object can include the first object 110. In an example, the magnetic coupling force can be provided using the first magnetic field. In an example, the first object can be held or fixed to a location on the tissue region using the magnetic coupling force. In an example, the object can be held or fixed to assist in a surgical or other medical procedure.

In an example, at 410, the magnetic coupling force between the first magnetic field source and the first object can be provided between the first magnetic field source and an intracorporeal apparatus. Examples of the intracorporeal apparatus can include an intracorporeal camera, scalpel, scissors, pliers, vacuum, or other surgical or medical apparatus.

At 415, the magnetic coupling force can be sensed and a resulting sensed force signal can be provided. In certain examples, the magnetic coupling force can be sensed using a force sensor (e.g., the force sensor 115). In an example, the resulting sensed force signal can include any information from the force sensor indicative of a sensed force, such as a property, characteristic, or other information provided by the force sensor. In an example, the magnetic coupling force can be sensed by suspending the first magnetic field source near the tissue region using a force sensor, such as a strain gauge or other force sensor. In other examples, the first magnetic field source can be suspended using a housing to create a space between the first magnetic field source and the tissue region.

At 420, the magnetic coupling force can be controlled using the sensed force signal to obtain a desired magnetic coupling force. In an example, the magnetic coupling force can be controlled to obtain the desired magnetic coupling force across a plurality of different tissue thicknesses. In certain examples, the magnetic coupling force can be controlled using a controller (e.g., the controller 120). In an example, the magnetic coupling force can be controlled by adjusting the first magnetic field produced by the first magnetic field source 105 (e.g., an electromagnet, a permanent magnet, or other magnetic field source) to obtain the desired magnetic coupling force. In other examples, the magnetic coupling force can be controlled by adjusting or controlling a distance between the first magnetic field source and the first object to obtain the desired magnetic coupling force, or the magnetic coupling force can be controlled by altering the magnetic susceptibility of the first object.

### Other Examples

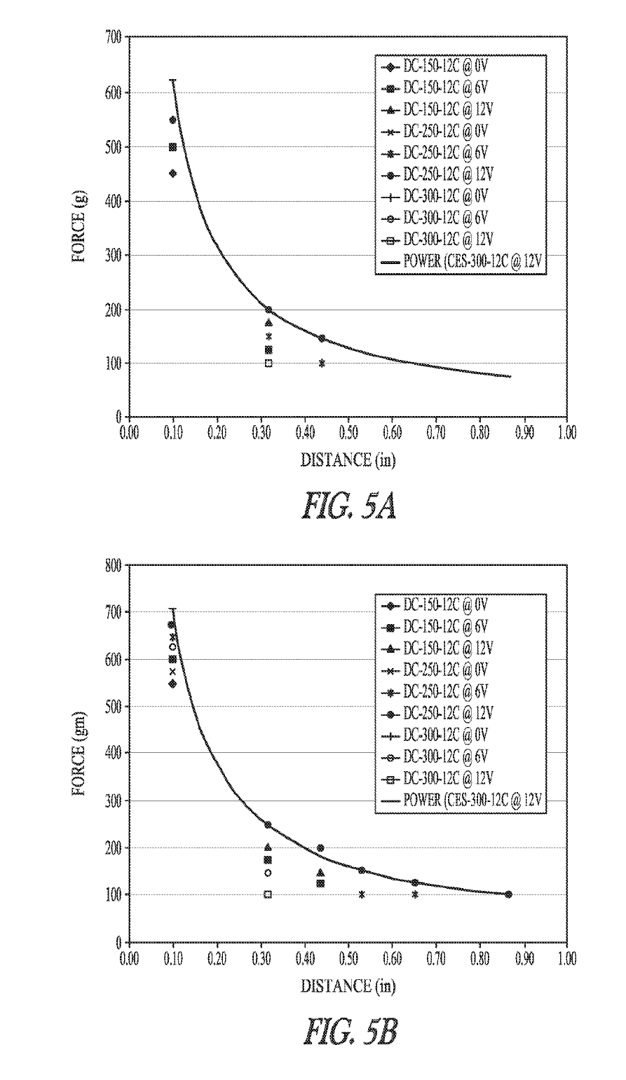

FIGS. 5A-5B illustrate generally examples of force relationships between three types of electromagnets and two types of fixed rare-earth magnets in a setup that replicates a proposed configuration for supporting instruments inside an abdominal cavity.

In this example, the relationship between three electromagnet configurations (DC-150-12C, DCA-250-12C, CEA-300-12C) and two fixed magnet configurations (0.375″Ø0×0.375″H, 0.375″Ø0×0.625″H) are shown, the electromagnets and fixed magnets physically separated by acrylic and delrin plates to a given height. The attractive force was measured by a spring scale at 0, 6 and 12 volts applied to the electromagnet, repeating this for each electromagnet and fixed magnet configuration at heights approximately 0.1″ to 0.9″.

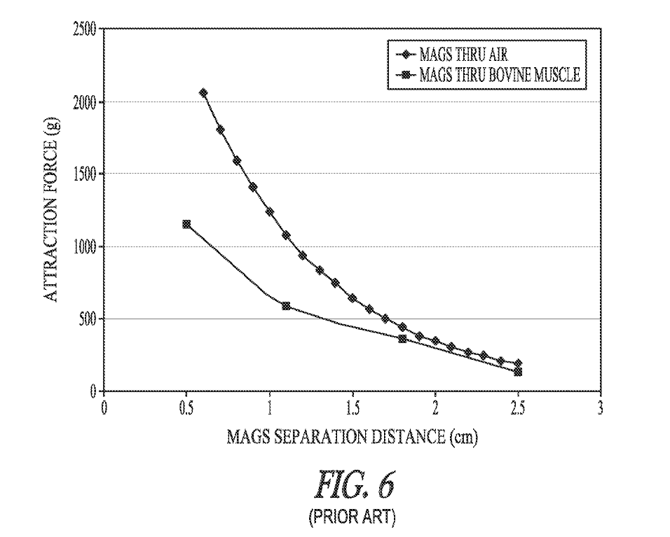

FIG. 6 illustrates generally an example of a relationship between an attraction force of two fixed magnets across varying separation distances through air and through tissue, as shown in Park et al, Trocar-less Instrumentation for Laparoscopy. Annals of Surgery Volume 245, Number 3, March 2007.

The electromagnet and fixed magnet configurations of FIGS. 5A and 5B can provide control of the attractive force between the electromagnets and the fixed magnets. However, in certain examples, control can vary from approximately 25% at close distances, up to 100% at large distances, due, at least in part, because certain unpowered electromagnets produce little to no measurable attractive force at distances larger than 0.5″. In an example, this relationship can be illustrated using a comparison of the information in FIGS. 5A and 5B with FIG. 6. Additionally, the electromagnet and fixed magnet configurations of FIGS. 5A and 5B supply about 33% of the force illustrated by the fixed magnets shown in FIG. 6.

In an example, the strength of the magnet configurations of FIGS. 5A and 5B can be increased by reconfiguring the fixed magnet into a loop configuration to reduce the line distance of the flux lines through air (e.g., using a horseshoe type magnet, as opposed to a disk magnet, such as that used above), or by improving the electromagnet design.

### Additional Notes

Although the above embodiments emphasize the first magnetic field source 105 as an external apparatus and the first object 110 as an intracorporeal apparatus, the first magnetic field source 105 can include or be coupled to an intracorporeal apparatus and the first object 110 can include an external apparatus.

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
a first magnetic field source configured to produce a first magnetic field across a tissue region, the first magnetic field providing a magnetic coupling force between the first magnetic field source and a first object;
a force sensor configured to sense the magnetic coupling force and to provide a resulting sensed force signal; and
a controller configured to receive the sensed force signal and to provide in response an output signal for controlling the magnetic coupling force to obtain a desired magnetic coupling force.

2. The system of claim 1, including the first object, the first object including a magnetic field source or receiver configured to provide a magnetic field or a magnetic susceptibility to obtain the magnetic coupling force.

3. The system of claim 2, wherein the first object includes or is coupled to an intracorporeal apparatus.

4. The system of claim 1, wherein the first magnetic field source includes a first electromagnet configured to produce the first magnetic field.

5. The system of claim 4, wherein the output signal is configured to adjust the first magnetic field produced by the first electromagnet to obtain the desired magnetic coupling force.

6. The system of claim 1, wherein the first magnetic field source includes a first permanent magnet.

7. The system of claim 1, wherein the output signal is configured to control a distance between the first magnetic field source and the first object to obtain the desired magnetic coupling force.

8. The system of claim 1, including a mount configured to suspend the first magnetic field source near the tissue region.

9. The system of claim 8, wherein the mount is configured to use at least part of the force sensor to suspend the first magnetic field source near the tissue region.

10. The system of claim 9, wherein the force sensor includes a strain gauge.

11. The system of claim 9, wherein the mount is configured to obtain the desired magnetic coupling force by using the output signal to adjust a distance between the first magnetic field source and the first object.

12. The system of claim 1, wherein the first magnetic field source is configured to hold the first object to a location on tissue region using the desired magnetic coupling force.

13. The system of claim 1, wherein the controller is configured to adjust the output signal to obtain the desired magnetic coupling force across a plurality of different tissue thicknesses.

14. A method comprising:
producing a first magnetic field across a tissue region using a first magnetic field source;
providing a magnetic coupling force between the first magnetic field source and a first object using the first magnetic field, the first object providing a magnetic field or providing a magnetic susceptibility to obtain the magnetic coupling force;
sensing the magnetic coupling force and providing a resulting sensed force signal; and
controlling the magnetic coupling force using the sensed forced signal to obtain a desired magnetic coupling force.

15. The method of claim 14, wherein the providing the magnetic coupling force between the first magnetic field source and the first object includes providing a magnetic coupling force between the first magnetic field source and an intracorporeal apparatus.

16. The method of claim 14, wherein the producing the first magnetic field using the first magnetic field source includes using a first electromagnet.

17. The method of claim 16, wherein the controlling the magnetic coupling force includes adjusting the first magnetic field produced by the electromagnet to obtain the desired magnetic coupling force.

18. The method of claim 14, wherein the producing the first magnetic field using the first magnetic field source includes using a first permanent magnet.

19. The method of claim 14, wherein the controlling the magnetic coupling force includes adjusting a distance between the first magnetic field source and the first object to obtain the desired magnetic coupling force.

20. The method of claim 14, wherein the sensing the magnetic field source includes suspending the first magnetic field source near the tissue region using a strain gauge.

21. The method of claim 14, including fixing the first object to a location on the tissue region using the magnetic coupling force.

22. The method of claim 14, wherein the controlling the magnetic coupling force to obtain the desired magnetic coupling force includes maintaining the desired magnetic coupling force across a plurality of different tissue thicknesses.
